(19) United States
(12) Patent Application Publication
(10) Pub. No.: US 2026/0232044 A1
(43) Pub. Date: Aug. 13, 2026

(54) INHALABLE SUBSTANCE CESSATION DEVICE

(71) Applicant: Qwytt Inc., San Jose, CA (US)

(72) Inventor: Joseph Cesar Perez, San Jose, CA (US)

(21) Appl. No.: 19/156,217

(22) PCT Filed: Feb. 13, 2024
(86) PCT No.: PCT/US2024/015600

§ 371 Date: Aug. 13, 2025

Related U.S. Application Data
(60) Provisional application No. 63/445,938, filed on Feb. 15, 2023.

(51) Int. Cl. A24F 47/00; A24F 40/42; A24F 40/485; A24F 40/53; A24F 40/60; A24F 40/65

(52) U.S. Cl. CPC A24F 47/00; A24F 40/485; A24F 40/53; A24F 40/60; A24F 40/65; A24F 40/42

(57) ABSTRACT

Systems and methods to provide real-time inhalable substances cessation support through the use of a wireless tracker 2024/173401 that collects data from a user's vaping habits and uses the data to create customized inhalable substances cessation programs. The wireless tracker may be a device configured to be attached to a mouthpiece of a vaping device. The wireless tracker may comprise one or more pressure sensors configured to detect an inhalation event and record information corresponding to the detected event. The system may also be configured to provide real-time feedback and guidance to the user based on their tracked usage.

### CROSS REFERENCE TO RELATED APPLICATIONS

This application is a non-provisional application which claims the benefit of priority of U.S. Provisional Application No. 63/445,938 filed on Feb. 15, 2023, which is hereby incorporated by reference in their entirety and should be considered a part of this specification.

### FIELD

The present invention relates generally to systems and methods to aid in the cessation of inhalable substances through guidance based on analysis of habits and usage of a user.

### BACKGROUND

Conventional systems or devices do not provide any real-time guidance or feedback to the user on their inhalable substance consumption or cessation progress. Users are often left to rely on their own self-control and motivation to reduce or quit inhalable substance use. There is a need for a system or device that provides real-time inhalable substances cessation guidance to help users quit using inhalable substances. Examples of inhalable substances include nicotine, synthetic nicotine, tobacco derived substances, cannabis, synthetic cannabinoids, flavored chemicals, inhalants, aerosols, gases, nitrites, solvents, smoke and/or vapor.

### SUMMARY

The system and methods described herein provide for an inhalation monitoring device. The inhalation monitoring device may comprise a body, the body may have a first opening at a first end and a second opening at a second end. The inhalation monitoring device may further have an enclosure comprising an air flow detector, a wireless transceiver, a processor electronically coupled to the wireless transceiver, a data storage device and a battery. The device may further comprise an attachment mechanism configured to attach the monitoring device to a smoking device or a vaping device. The enclosure may be fixed or integral to the body.

In some embodiments, the second opening may be configured to attach to and create a seal around a mouthpiece of the smoking device or the vaping device.

In some embodiments, the attachment mechanism may be a band connectable to the second end, the attachment band may be sized to secure the monitoring device to an atomizer cartridge of the smoking device or vaping device.

In some embodiments, the device may further comprise a plurality of visual indicators disposed on the enclosure, the visual indicators may comprise light emitting diodes.

In some embodiments, the processor may be configured to perform the operations of displaying, via one or more of the plurality of visual indicators, an indication of an inhalation status. The processor may further be configured to perform the operations of determining one or more inhalations via the airflow detector and storing data on the data storage device about the one or more inhalations.

In some embodiments, the processor may further be configured to perform the operations of receiving from a client device, inhalation goal data comprising data about allowable inhalations for a time period and determining whether a count of the one or more inhalations are within an allowed inhalation limit for the time period.

In some embodiments, the processor may further be configured to perform the operations of determining a first inhalation and determining a time or interval for a next inhalation.

In some embodiments, the processor may be configured to perform the operations of transmitting, via the transceiver, to a client device, inhalation data comprising inhalation counts and time information for each count.

In some embodiments, the body may have a channel formed therethrough connected to the first opening and the second opening, with the air flow detector positioned about the body to detect air flow through the channel.

The system and methods described herein may further provide for a inhalable substances cessation system. The inhalable substances cessation system may comprise one or more servers, an application operable on a user device and a monitoring unit.

The monitoring unit may comprise an enclosure, a processing unit, a memory module, a datastore module, one or more pressure sensors, a communication module, a battery module, one or more indicators, a mouthpiece and an adapter module. In some embodiments, the monitoring unit may be configured to physically attach to a smoking device or vaping device, wherein the attaching creates an airtight interface between the adapter module of the monitoring unit and the smoking device or vaping device. The monitoring unit may further be configured to collect usage data, wherein the collecting may comprise identifying one or more puffs taken by the user. The identifying of puffs may be based on detecting changes in pressure at the one or more pressure sensors. The monitoring unit may further be configured to record a timestamp of each of the one or more puffs, record a duration of each of the one or more puffs and calculate a volume of each of the one or more puffs based on the duration of the puff and the detected change in pressure at the one or more pressure sensors.

In some embodiments, the monitoring unit may further be configured to generate a user profile. The user profile may be based at least in part on usage data collected over a predetermined period of time. The user profile may comprise an average daily number of puffs, an average puff volume, an average puff duration, an average total daily usage, a maximum flow rate, a flow rate average and/or an average time between puffs.

In some embodiments, the monitoring unit may further be configured to calculate a goal profile. The goal profile may be based on the user profile. The goal profile may comprise a goal usage value, wherein the goal usage value corresponds to a total duration of daily use or a total volume of daily use, a number of puffs goal and a wait time goal. The wait time goal may correspond to a time between puffs.

In some embodiments, the collected usage data may be transmitted to the user device, and the user device may perform the generation of the user profile and the calculation of the goal profile.

In some embodiments, the user device may be configured to display information corresponding to the goal profile and current usage data, wherein the current usage data comprises a current number of puffs, current duration of use and a current usage volume.

In some embodiments, the information displayed on the user device may comprise the goal usage value, the current duration of use or the current usage volume, the number of puffs goal, the current number of puffs and a countdown timer. The countdown timer may be configured to display an amount of time until a next puff.

In some embodiments, the countdown timer may be reset after detection of each puff.

In some embodiments, the system may further be configured to generate a user adherence metric corresponding to how closely the current usage data matches the goal profile.

In some embodiments, the goal profile may be updated based on collected usage data over a sliding window. The goal profile may be calculated by a trained machine learning model. The trained machine learning model may be a deep learning time series model. The trained machine learning model may be trained on a training set comprising of collected usage data from one or more users, user profiles of one or more users, goal profiles of one or more users and user adherence metrics of one or more users.

In some embodiments, the one or more indicators of the monitoring unit may be light emitting diodes (LEDs).

While the disclosure describes a system that is attachable to other smoking or vaping devices. The structures and functionality described herein may be integrally formed with such smoking or vaping devices. For example, one or more components or structures of the cessation device may be integrally formed with a smoking or vaping device.

The appended claims may also serve as a summary of this application.

The features and components of these embodiments will be described in further detail in the description which follows. Additional features and advantages will also be set forth in the description which follows, and in part will be implicit from the description, or may be learned by the practice of the embodiments. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1 is a diagram illustrating an exemplary environment in which some embodiments may operate.

FIG. 2A is a diagram illustrating an exemplary client device in accordance with aspects of the present disclosure.

FIG. 2B is a diagram illustrating an exemplary server in accordance with aspects of the present disclosure.

FIG. 2C is a diagram illustrating an exemplary cessation device in accordance with aspects of the present disclosure.

FIG. 3A is a diagram illustrating an exemplary cessation device in accordance with some embodiments.

FIG. 3B is a diagram illustrating an exemplary cessation device in accordance with some embodiments.

FIG. 3C is a diagram illustrating an exemplary cessation device in accordance with some embodiments.

FIG. 3D is a diagram illustrating an exemplary cessation device in accordance with some embodiments.

FIG. 3E is a diagram illustrating an exemplary cessation device in accordance with some embodiments.

FIG. 3F is a diagram illustrating an exemplary cessation device in accordance with some embodiments.

FIG. 3G is a diagram illustrating an exemplary cessation device in accordance with some embodiments.

FIG. 3H is a diagram illustrating an exemplary cessation device in accordance with some embodiments.

FIG. 3I is a diagram illustrating an exemplary cessation device in accordance with some embodiments.

FIG. 3J is a diagram illustrating an exemplary cessation device in accordance with some embodiments.

FIG. 3K is a diagram illustrating an exemplary cessation device in accordance with some embodiments.

FIG. 3L is a diagram illustrating an exemplary cessation device in accordance with some embodiments.

FIG. 3M is a diagram illustrating an exemplary cessation device in accordance with some embodiments.

FIG. 4A is a flow chart illustrating an exemplary method that may be performed in accordance with some embodiments.

FIG. 4B is a flow chart illustrating an exemplary method that may be performed in accordance with some embodiments.

FIG. 4C is a flow chart illustrating an exemplary method that may be performed in accordance with some embodiments.

FIG. 5 is a flow chart illustrating an exemplary method that may be performed in accordance with some embodiments.

FIG. 6 is a flow chart illustrating an exemplary method that may be performed in accordance with some embodiments.

FIG. 7 is a diagram illustrating an exemplary computer/control system that may perform processing in some embodiments and in accordance with aspects of the present disclosure.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

The following generally relates to a system and methods for real-time inhalable substances cessation guidance. The real-time inhalable substances cessation guidance system may comprise a cessation device, one or more servers, one or more client devices, one or more applications operating on the one or more servers and/or one or more client devices, one or more trained machine learning models configured to generate one or more cessation guidance programs and one or more user interface modules configured to display visual representations of the generated cessation guidance programs. The user interface modules may further be configured to display current usage information captured by the cessation devices, historic usage information, user progress and/or combination thereof.

In some embodiments of the present invention, a wireless vaping device is provided that includes built-in LED indicators for providing real-time inhalable substances cessation guidance to the user. The cessation guidance may be personalized for each individual user and generated in real-time, in response to the user's usage habits, characteristics, inhalable substances consumption patterns and user preferences.

In some embodiments, the cessation device may be configured to attach to a vaping device of the user to provide wireless tracking of usage of the vaping device. The cessation device may further comprise an attachment member configured to rigidly connect the cessation device with the vaping device. In some embodiments, a strap or arm may extend from a first side of the cessation device, wrap around a portion of the vaping device and attach to a second side of the cessation device, wherein the second side is opposite to that of the first side. In some embodiments, the cessation device may be configured to connect with a plurality of different vaping devices. In some embodiments, one or more adapting members may be used to couple the cessation device to a vaping device. The cessation device may be configured to provide for quick and easy attachment, removal and reattachment to a wide range of vaping devices.

In some embodiments, the cessation device may be integrated with the vaping device. For example, the vaping device itself may include a cessation module, one or more pressure sensors, flow sensors, fluid level sensors and/or power sensors. The cessation module may be configured track/monitor/record the user's usage/consumption/habits based on data collected from the one or more sensors. This exemplary vaping device with an integrated cessation module and sensors may perform all the methods and functionality of the cessation device as described herewithin.

In some embodiments, the device may comprise one or more sensors that detect when the user is inhaling and wireless communication capabilities for sending data to a connected multiplatform application operating on a client device. In some embodiments, the one or more sensors may be one or more pressure sensors. In some embodiments, the one or more sensors may be a power sensor configured to detect the activation of the vaping device. In some embodiments, the one or more sensors may be a combination of pressure sensors and power sensors. In some embodiments, the cessation device may be configured to collect data on the user's vaping habits, including the frequency, duration, and intensity of use. Data associated with the detection of the user's inhalation may be collected and sent to a connected multiplatform application operating on the client device.

In some embodiments, an example operation of the system may start with the user inhaling through the cessation device. The one or more pressure or power sensors may detect the inhalation and begin to collect information corresponding to the inhalation event (puff). The cessation device may then send the collected data to the client device, which may then analyze the data and provide real-time feedback to the user.

In some embodiments, the system may be configured to analyze the received data to create customized inhalable substances cessation guidance programs for the user. The customized inhalable substances cessation guidance programs may be personalized for each user. The personalized guidance programs may be generated by one or more artificial intelligence (AI) models and/or machine learning (ML) models. In some embodiments, the AI models and/or ML models may be models trained on datasets of historic user data from a plurality of users. The one or more AI/ML models may be used by the system to create one or more optimized cessation guidance programs for the user. The AI/ML models may be configured to take into account factors such as the user's vaping frequency, duration of use, and time of day when generating cessation guidance programs for the user.

In some embodiments, the system may be configured to provide the user with real-time feedback through indicator lights (LEDs) disposed on an externally facing wall of the device. The indicator lights may be configured to provide visual cues to the user to let them know whether they are meeting their goals or whether they should modify their inhalable substances consumption patterns. The LEDs may communicate information related to the user's current usage, their adherence to the guidance program, and/or usage goals set by the guidance program. In some embodiments, the indicator lights may be illuminated in a manner to communicate to the user whether another puff is advisable based on their cessation program and their current usage data. In some embodiments, the LEDs may be configured to change color to indicate whether inhalable substances use is advisable based on the user's cessation program.

In some embodiments, a user interface operating on the client device of the user may be configured to display visualizations corresponding their vaping data, their progress, and cessation guidance program goals. In some embodiments, the user may adjust their cessation guidance program as needed. Real-time feedback and guidance may also be provided to the user through visualizations displayed in the user interface.

In some embodiments, the system is further configured to allow the user to create or join groups for support and accountability. In some embodiments, the device may be used in both administrative and casual settings, with either designated users able to provide oversight or all members able to keep each other accountable and compete to reach their goals together.

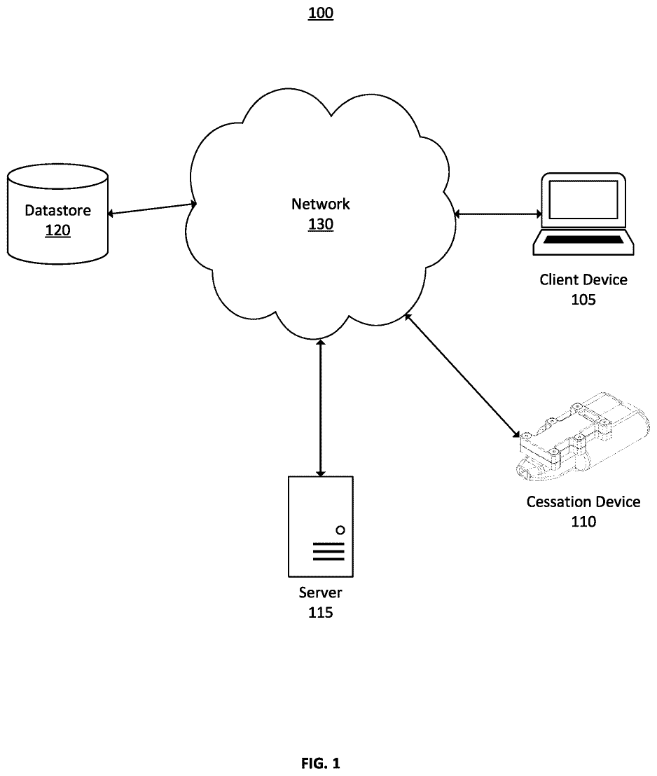

FIG. 1 is a diagram illustrating an exemplary a inhalable substances cessation system 100 in which some embodiments may operate. The inhalable substances cessation system 100 may comprise one or more client devices 105, one or more cessation devices 110, one or more servers 115, one or more datastores 120 and one or more networks 130.

The client devices 105 may be any computing device capable of communicating over network 130. The client devices 105 may be integrated into a notebook computer, smartphone, personal digital assistant, desktop computer, tablet computer, or other computing device.

Cessation device 110 may be configured to attach to a vaping device, detect operation of the vaping device, and collect information related to usage of the vaping device. In some embodiments, the detection of the operation of the vaping device may be based on readings collected from one or more pressure sensor and/or one or more power sensors. In some embodiments, the cessation device may be configured to determine a duration of an inhalation event and an amount of inhalable substances inhaled during the inhalation event. The amount of inhalable substances inhaled may be calculated based on pressure readings from the one or more pressure sensors and the duration of the inhalation event. Both raw sensor readings and calculated values corresponding to inhalation events may be stored on the cessation device 110 and/or transmitted to client devices 105 and/or servers 115. Cessation device 110 may further comprise LED indicator lights configured to provide real-time visual feedback and guidance to the user.

Server 115 may be one or more physical or virtual machines configured to communicate with the one or more client devices 105, one or more cessation devices 110 and the one or more datastores 120. The one or more servers 115 may be configured as a distributed computing infrastructure and processing of applications and other software may be carried out on the cloud.

Datastores 120 may communicate with one another over network 130. Datastores 120 may be any storage device capable of storing data for processing or as a result of processing information at the client devices 105, cessation devices 110 and/or servers 115. The Datastores 120 may be a separate device or the same device as server 115. The Datastores 120 may be located in the same location as that of server 115, or at separate locations.

Network 130 may be an intranet, internet, mesh, LTE, GSM, peer-to-peer or other communication network that allows the one or more servers 115 to communicate with the one or more client devices 105, one or more cessation devices 110 and Datastores 120. In some embodiments, network 130 may further include Internet of Things (IoT) networks such as LORAWAN, Zigbee and BLE.

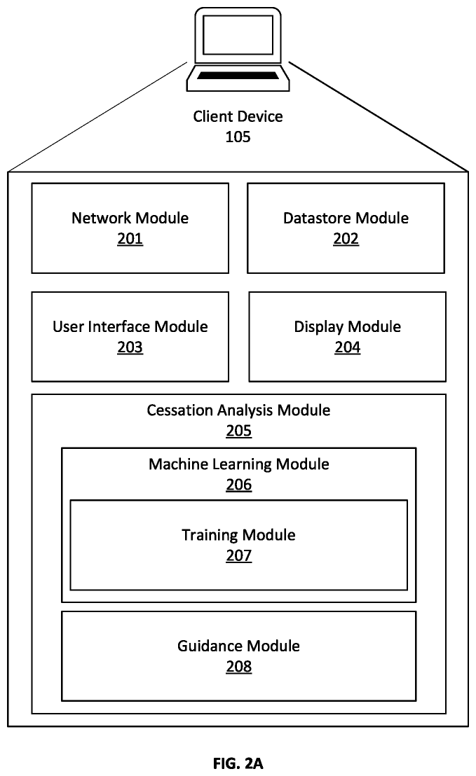

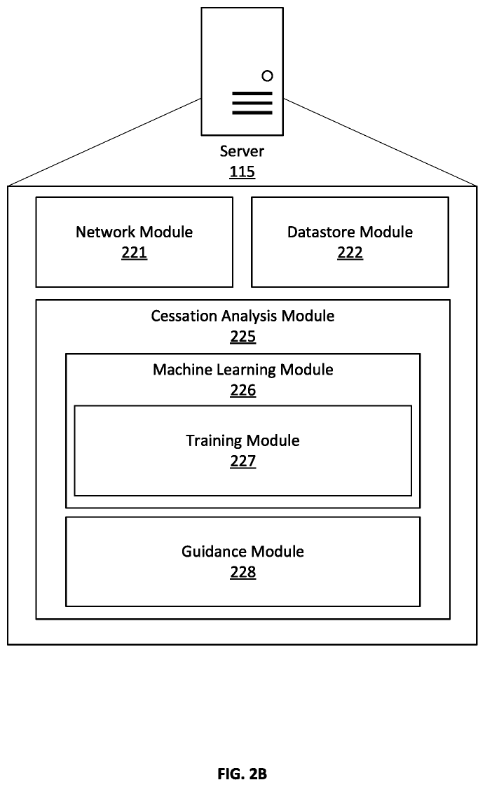

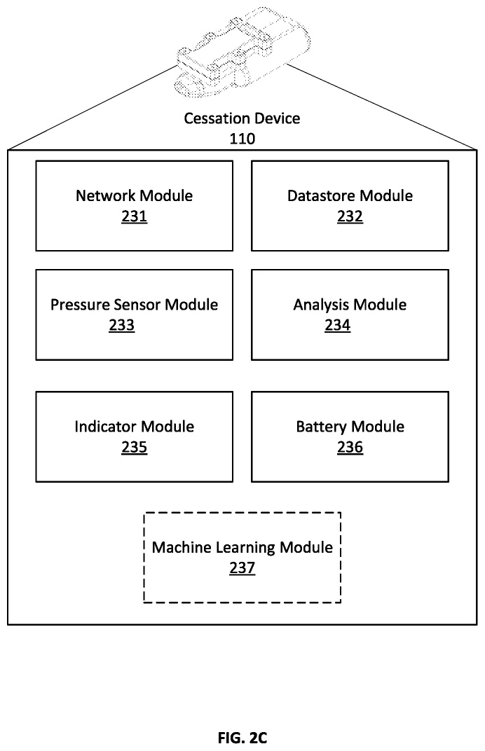

FIG. 2A is a diagram illustrating an exemplary client device 105 in accordance with aspects of the present disclosure. Client device 105 may comprise network module 201, datastore module 202, user interface module 203, display module 204, and cessation analysis module 205.

Network module 201 may transmit and receive data from other computing systems via a network. In some embodiments, the network module 201 may enable transmitting and receiving data from the Internet. Data received by the network module 201 may be used by the other modules. The modules may transmit data through the network module 201. In some embodiments, network module may facilitate communication between the client device 105 and cessation device 110 through Bluetooth, BLE, WIFI, ZIGBEE or other internet of things (IoT) networks.

The datastore module 202 may be configured to store information generated by the one or more modules operating on the client device 105. The one or more modules operating on the client device 105 may also retrieve information from the datastore module 202. In some embodiments, the datastore module 202 may be configured to store information received from cessation device 110. Datastore module 202 may store raw and/or processed information.

User interface module 203 may generate a user interface (UI) based on information received from cessation device 110 and/or server 115. The user interface module 203 may further manage user interactions with the UI and the updating of the UI based at least in part on the user interactions. The UI may comprise one or more interfaces and/or elements of the one or more interfaces. The one or more interfaces may include panels, windows, tabs, containers and/or combination thereof. The one or more interfaces may be any structure or object that organizes and/or displays information graphically. In some embodiments, the user interface module 203 may be configured to manage a UI generated remotely on server 115. The managing may comprise receiving user interactions with the UI, processing the user interactions, updating the UI based on the user interactions, data received from cessation device 110 and/or information received from server 115, transmit the user interactions to the server 115 and transmitting information associated with the user interface module 203 processing the user interactions.

Display module 204 may be any device configured to display graphical representations of information (LCD display, OLED display, DLP display, etc.). Display module 204 may be further configured to display graphical representations of the UI generated or received by the user interface module 203. Display module 204 may further be configured to display interactions of the user with the UI.

Cessation analysis module 205 may further comprise machine learning module 206, training module 207 and guidance module 208. Cessation analysis module 205 may be configured track the progress of a user and adjust guidance and recommendations provided to the user based on received cessation device usage data, historic cessation device usage data and one or more cessation guidance programs generated for the user.

Machine learning module 206 may further comprise training module 207. Machine learning module 206 may be configured to generate one or more machine learning models. The one or more machine learning models may be used to analyze cessation device 110 usage data. The analysis performed by the one or more machine learning models may be used by the guidance module 208 in the generation of cessation guidance plans for a user. In some embodiments, the machine learning module may be configured to analyze interaction of the user with the user interface on the client device 105 along with usage/consumption habits of the user received from the cessation device 110 to generate one or more indications of adherence to the generated guidance and to update the guidance plan in real-time.

Machine learning module 206 may be configured to generate one or more AI models and/or one or more ML models based on training performed by training module 207. The training module 207 may be trained based on training data comprising historic usage data for a plurality of users, historic cessation guidance programs generated for the plurality of users and adherence metrics corresponding to historic cessation guidance programs for the plurality of users. In some embodiments, the one or more generated AI/ML models may be Deep Learning Time Series (DLTS) models, Artificial Neural Networks (ANNs), Recurrent Neural Networks (RNNs), Radial Basis Functions Neural Networks (RBFNNs), Bayesian Neural Networks (BNNs), Generalized Regression Neural Networks (GRNNs), Convolutional Neural Networks (CNNs), CART Regression Trees (CARTs), Support Vector Regression (SVR) models, Long Short-term Memory (LSTM) models, Autoregressive Moving Average (ARMA) models, Autoregressive Integrated Moving Average (ARIMA) models, Seasonal Autoregressive Integrated Moving-Average (SARIMA) models, Linear Regression models, Multi-Layer Perceptron (MLP) models, Random Forest models, Gradient Boosting models, Gated Recurrent Unit (GRU) models or combination thereof. Other AI/ML models may also be generated.

In some embodiments, training may be performed to generate one or more new models or update a previously generated model. In some embodiments, the training module may receive an updated training set for retraining after the initial training of the model is performed.

FIG. 2B is a diagram illustrating an exemplary server 115 in accordance with aspects of the present disclosure. Server 115 may comprise network module 221, datastore module 222 and Cessation analysis module 225.

Network module 221, datastore module 222 and cessation analysis module 225, machine learning module 226, training module 227, and guidance module 228 may be the same or similar to that of network module 201, datastore module 202 and cessation analysis module 205, machine learning module 206, training module 207 and guidance module 208 in FIG. 2A and will not be described for the sake of brevity.

FIG. 2C is a diagram illustrating an exemplary cessation device 110 in accordance with aspects of the present disclosure. Cessation device 110 may comprise network module 231, datastore module 232, pressure sensor module 233, analysis module 234, indicator module 235 and battery module 236. Network module 231 and datastore module 232 may be the same or similar to that of network module 201 and datastore module 202 in FIG. 2A.

In some embodiments, the exemplary cessation device 110 optionally includes a machine learning module 237 (similar to machine learning module 206). The machine learning module 237 may operate on the exemplary cessation device 110.

Pressure sensor module 233 may comprise one or more sensors configured to measure pressure and/or change in pressures during operation of the cessation device by the user. In some embodiments, the pressure sensor module may also comprise one or more flow sensors. The one or more pressure sensors may measure absolute pressure, differential pressure, gauge pressure or combination thereof. In some embodiments, the one or more pressure sensors of the pressure sensor module 233 may employ one or more sensing principles, wherein the one or more sensing principles comprises Resistive, Capacitive, Piezoelectric, Optical and/or MEMS based sensing.

Analysis module 234 may be configured to receive data measured by the pressure sensor 233 and the one or more cessation guidance programs generated by the guidance module 208 of client device 105. The one or more cessation guidance programs may also be received from server 115 (generated by guidance module 228). In some embodiments, the analysis module 234 may be configured to identify inhalation events based on the data received from the pressure sensor module 233, calculate usage information based on the identified inhalation event and the received pressure sensor module data, track and store usage information captured by the cessation device, determine a level of adherence to the one or more cessation guidance programs for the user and determine additional guidance to be communicated to the user based on the adherence. The analysis module 234 may also be configured to instruct the indicator module 235 to provide real-time feedback to the user by performing one or more preconfigured illumination procedures. The one or more preconfigured illumination procedures may be based on the calculated/tracked usage, level of adherence to the guidance program, and additional guidance for the user as determined by the analysis module 233. In some embodiments, the indicator module 235 may drive the illumination of one or more LEDs based on the information being conveyed. In some embodiments, the color of illumination, duration of illumination, temporal pattern of illumination, spatial pattern of illumination or combination thereof may be used to convey information. For example, red flashing LEDs may indicate poor adherence to the guidance program, while a solid green light may correspond to meeting or exceeding the guidance goals.

Battery module 236 may comprise one or more rechargeable or non-rechargeable batteries. The battery module may be any device capable of providing electrical power to the cessation device.

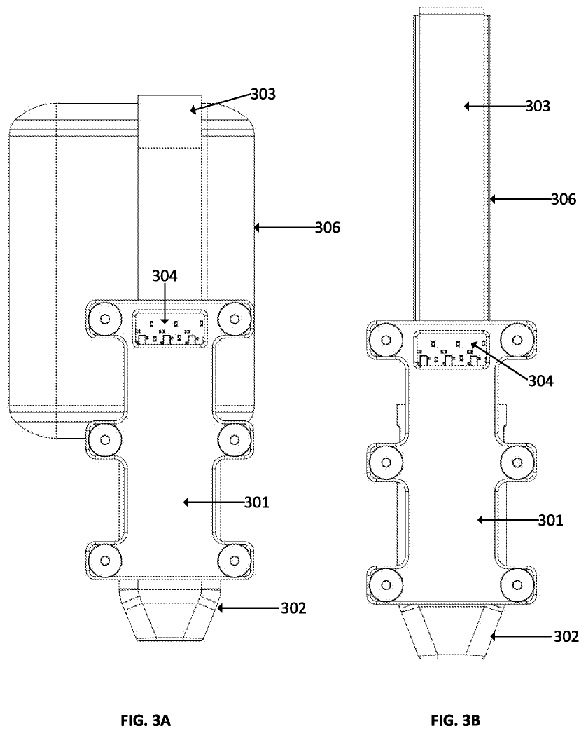

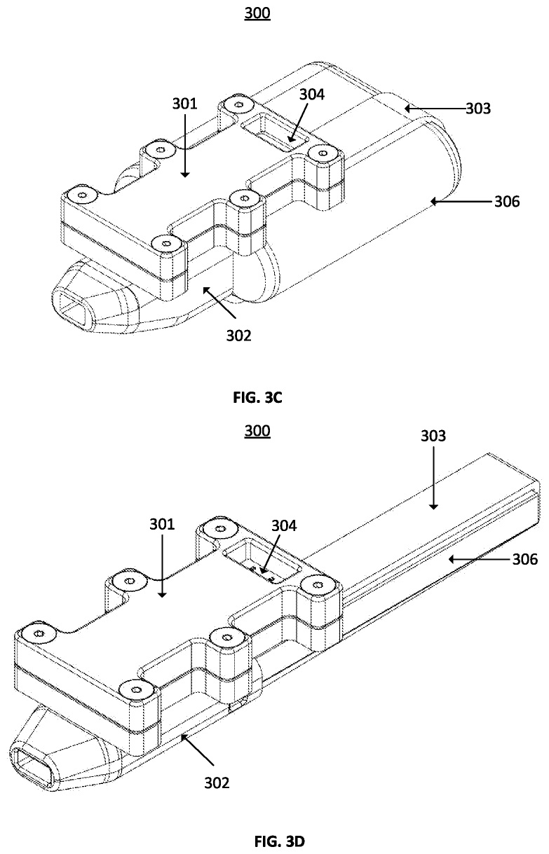

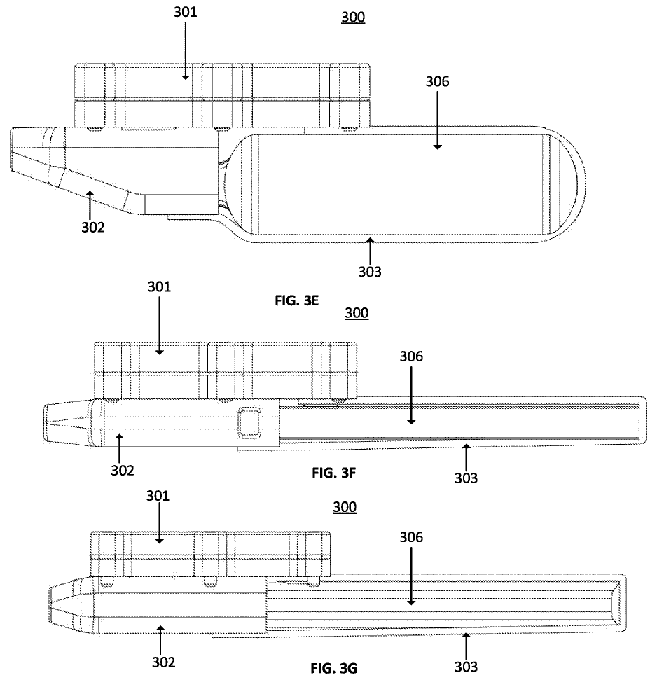

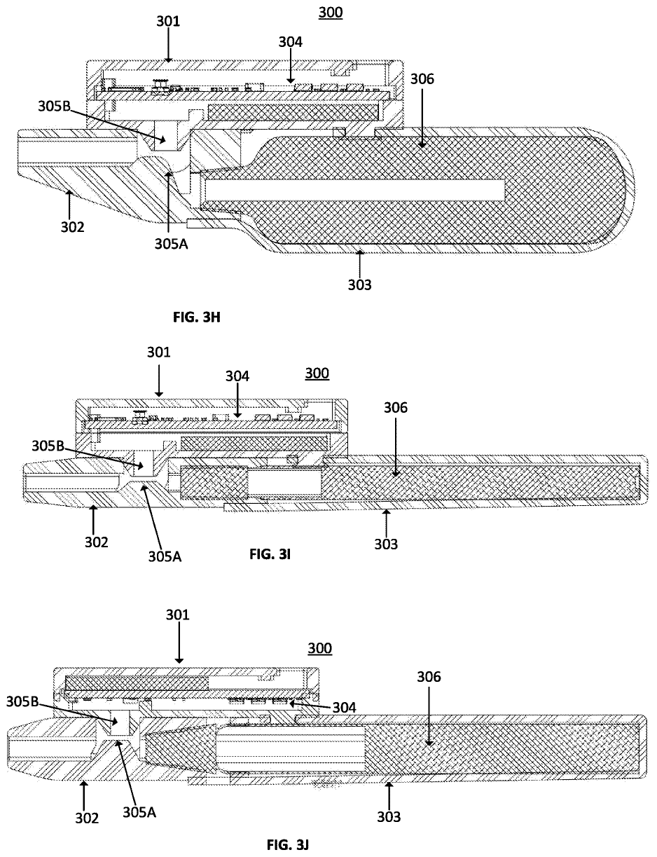

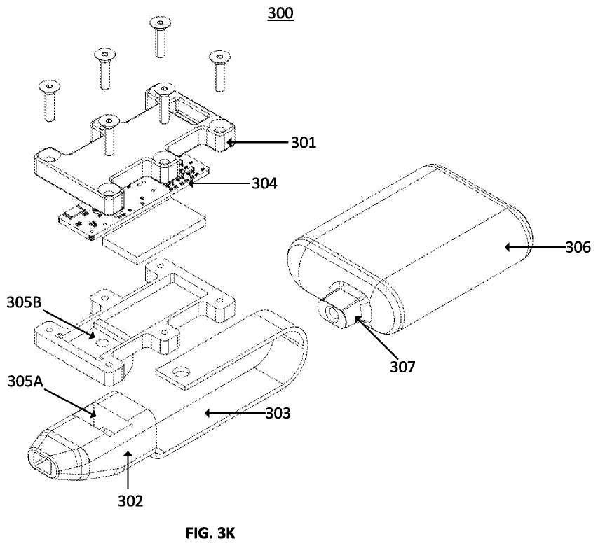

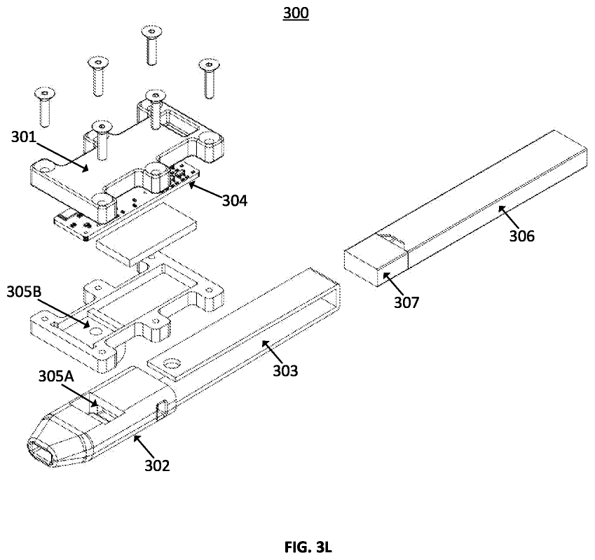

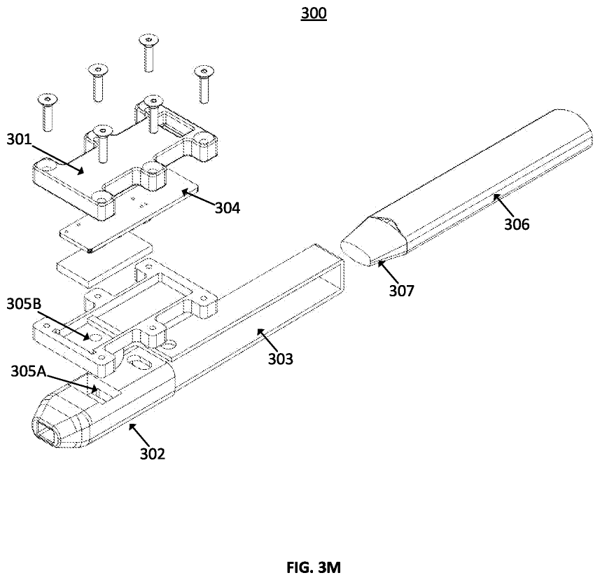

FIGS. 3A-3M are diagrams illustrating exemplary cessation devices 300 in accordance with some embodiments. FIGS. 3A and 3B are top views of exemplary cessation devices in accordance with some embodiments of the current invention. FIGS. 3C and 3D are isometric views of exemplary cessation devices in accordance with some embodiments. Cessation device 300 may comprise may sensor enclosure 301, mouthpiece cover 302, strap 303 and analysis unit 304. As shown in FIGS. 3A-3G , cessation device 300 has been installed on vaping device 306. Strap 303 may be configured to hold vaping device 306 in contact with cessation device 300. The strap 303 may be configured to apply a force to the vaping device 306 sufficient to create an airtight seal at the interface between the mouthpiece cover 302 of the cessation device and a mouthpiece portion of the vaping device 306. FIGS. 3E-3G are side views of exemplary cessation devices 300 in accordance with some embodiments.

FIGS. 3H-3J are cutaway side views corresponding to the side views of the exemplary cessation devices 300 shown in FIGS. 3E-3G. FIGS. 3H-3J further show pressure sensor channel 305A in the mouthpiece cover 302 and pressure sensor tube 305B in the sensor enclosure 301. In some embodiments, there may be one or more pressure sensor tubes 305B disposed in the pressure sensor channel 305A. In some embodiments, one or more pressure sensors tubes 305B may be disposed in the pressure sensor channel 305A in combination with one or more pressure sensor tubes positioned outside of the pressure sensor channel 305A. For example, the cessation device may have one or more dynamic pressure sensors connected to pressure sensor tubes 305B positioned in the pressure sensor channel 305A, and one or more static pressure sensors connected to pressure sensor tubes positioned externally to the mouthpiece enclosure, so as to measure atmospheric pressure.

FIG. 3K is an exploded isometric view of the exemplary cessation device of FIGS. 3E and 3H in accordance with some embodiments. FIG. K shows the vaping device mouthpiece 307 attached to the vaping device 306. The mouthpiece cover 302 of the cassation device 300 may be designed based on the shape and dimensions of the vaping device mouthpiece 307. This may allow for a snug, airtight fit between the two devices and provide an accurate measure of pressure differential during inhalation events captured by the cessation device 300.

FIG. 3L is an exploded isometric view of the exemplary cessation device of FIGS. 3F and 3I in accordance with some embodiments.

FIG. 3M is an exploded isometric view of the exemplary cessation device of FIGS. 3G and 3J in accordance with some embodiments.

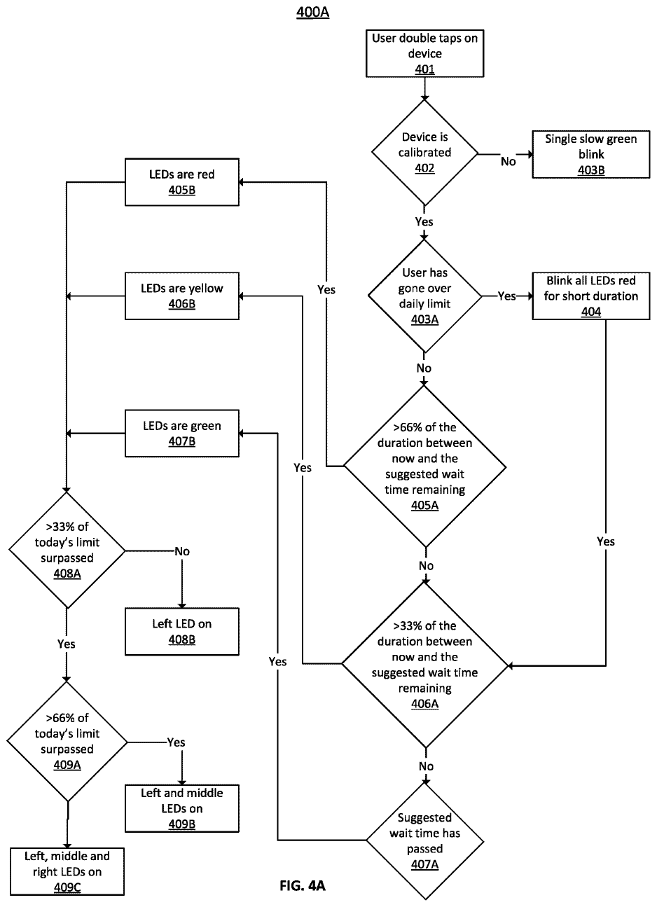

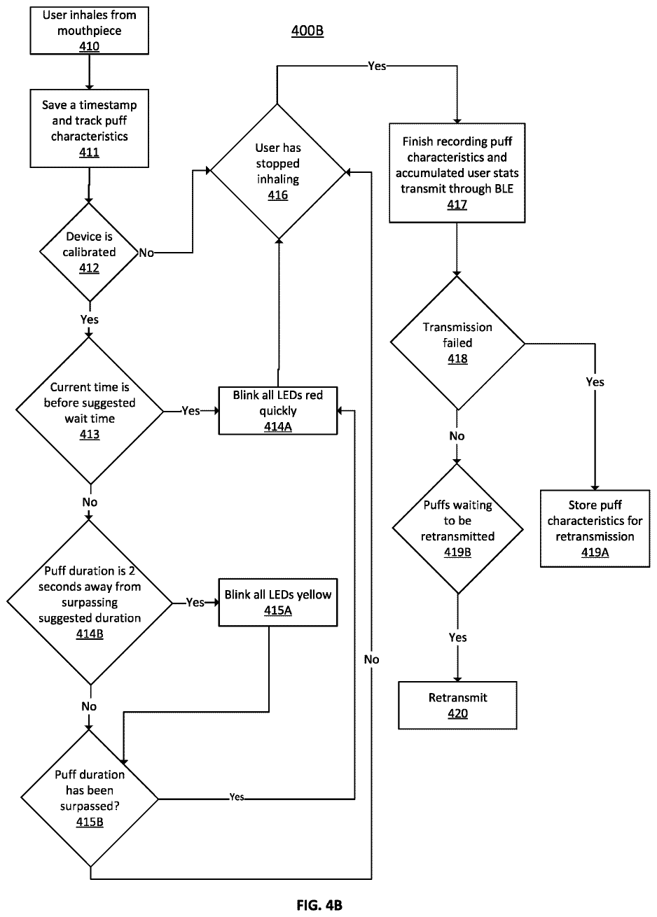

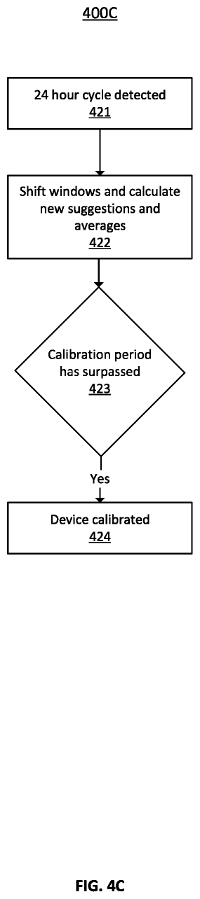

FIG. 4A is a flow chart illustrating an exemplary method 400A that may be performed in accordance with some embodiments.

At step 401, the cessation device 110 is configured to receive a double tap on the cessation device from the user.

At step 402, the cessation device determines if the device is calibrated. If the device is calibrated, the device proceeds to step 403A. If the device is not calibrated, the system proceeds to step 403B, where an indicator (LED) on the cessation device 105 is instructed to produce a single slow green blink.

At step 403A, the cessation device is configured to determine if the user has gone over their daily limit. If the daily limit has been exceeded, the cessation device proceeds to step 404, where all LED indicators are instructed to blink red for a short duration. If the daily limit has not been exceeded, the cessation device proceeds to step 405A.

At step 405A, the cessation device is configured to determine if a predetermined amount of the suggested wait time remains. For example, the predetermined amount of time may be a percentage of the suggested wait time duration. If the remaining duration from the current time is over the percentage of the suggested wait time, for example 66% of the duration remains, the cessation device proceeds to step 405B where the indicator LEDs are instructed to illuminate red. In some embodiments, step 405A may use a determination of the percentage of the duration that has already passed instead of the percentage of the duration that remains.

Cessation device is configured to proceed to step 406A if the remaining duration is below the predetermined amount of time. At step 406A, the cessation device is configured to determine if more than a second predetermined amount of the suggested wait time remains. For example, the second predetermined amount of time may be a smaller percentage of the suggested wait time duration than that used at step 405A. In some embodiments, the second predetermined amount of time may be more than 33% of the remaining duration from the current time. In this example, if the current remaining duration is more than or equal to 33% but under 66%, the cessation device proceeds to 406B where the indicator LEDs are instructed to illuminate yellow. If the current remaining duration is below the second predetermined amount of time, the cessation device proceeds to step 407A.

At step 407A, if the cessation device determines that the suggested wait time has passed, the cessation device proceeds to step 407B where the indicator LEDs are instructed to illuminate green.

After steps 405B, 406B and/or 407B, the cessation device proceeds to step 408A. At step 408A, the cessation device determines the amount of the day's limit that has already been used. If the usage of the user is below a first limit level the cessation device proceeds to step 408B where an indication as to the amount of usage left compared to the daily limit is displayed. For example, if the daily usage is below 33% of the daily limit a single LED may be illuminated. In some embodiments, the single illuminated LED may be a leftmost LED of a plurality of LEDs, and wherein the order and/or the number of illuminated LEDs corresponds to the remaining usage. If the usage is above the first limit level, the cessation device may proceed to step 409A.

At step 409A, if the usage of the user is below a second limit level but higher than the first limit level, the cessation device proceeds to step 408B where an indication as to the amount of usage left compared to the daily limit is displayed. For example, if the daily usage is above 33% but below 66% of the daily limit the left and middle LEDs may be illuminated.

If the usage of the user is above the second limit level the cessation device proceeds to step 408C where an indication as to the amount of usage left compared to the daily limit is displayed. For example, if the daily usage is above 66% of the daily limit, the left, middle and right LEDs may be illuminated.

FIG. 4B is a flow chart illustrating an exemplary method 400B that may be performed in accordance with some embodiments.

At step 410, the user performs a puff by inhaling through the mouthpiece.

At step 411, the cessation device is configured to save a timestamp of the puff and track the puff characteristics.

At step 412, The cessation device is configured to determine if the device is calibrated. If the device is calibrated, the device proceeds to step 413.

At step 413, the cessation device compares the current time with the suggested wait time. If the current time is before the suggested wait time, the cessation device proceeds to step 414A where all LEDs are instructed to quickly blink red. From step 414A, the cessation device proceeds to step 416, where the user has stopped inhaling. If the current time is after the suggested wait time, the cessation device proceeds to step 414B.

At step 414B, the cessation device is configured to determine if the duration of the puff is two seconds away from surpassing the suggested puff duration. If the determination is true, the cessation device proceeds to step 415A where all LEDs are instructed to quickly blink Yellow. If the determination is false, the cessation device proceeds to step 415B.

At step 415B, the cessation device is configured to determine if the puff duration has been surpassed. If the puff duration has been surpassed, the cessation device proceeds to step 414A where all LEDs are instructed to quickly blink red. If the puff duration has not been surpassed the cessation device proceeds to step 416, where the user has stopped inhaling.

From step 416, where the user has stopped inhaling, the cessation device proceeds to step 417.

At step 417, the cessation device finishes recording the puff characteristics and accumulated user stats. The recorded information is then transmitted through BLE.

At step 418, the cessation device is configured to determine if the transmission of the recorded information has failed. If the transmission of the information has failed, the cessation device proceeds to step 419A. If the transmission of the information has not failed, the cessation device proceeds to step 419B.

At step 419A, the cessation device is configured to store the puff characteristics for retransmission at a later time.

At step 419B, the cessation device is configured to determine if there are puffs where the puff information is waiting to be retransmitted. If there are puffs waiting to be retransmitted, the cessation device proceeds to step 420 where the puffs are then retransmitted.

FIG. 4C is a flow chart illustrating an exemplary method 400C that may be performed in accordance with some embodiments.

At step 421, a 24 hour cycle is detected by the cessation device.

At step 422, the cessation device is configured to shirt the time window and calculate new suggestions and averages for the user.

At step 423, the cessation device checks to see if the calibration period has ended. If the calibration period has ended, the cessation device proceeds to step 424 where the device calibration is performed.

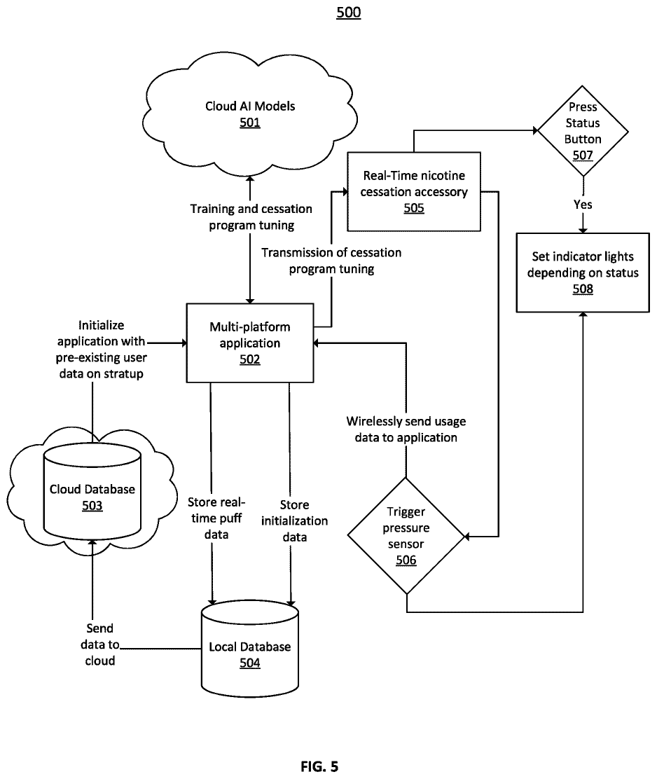

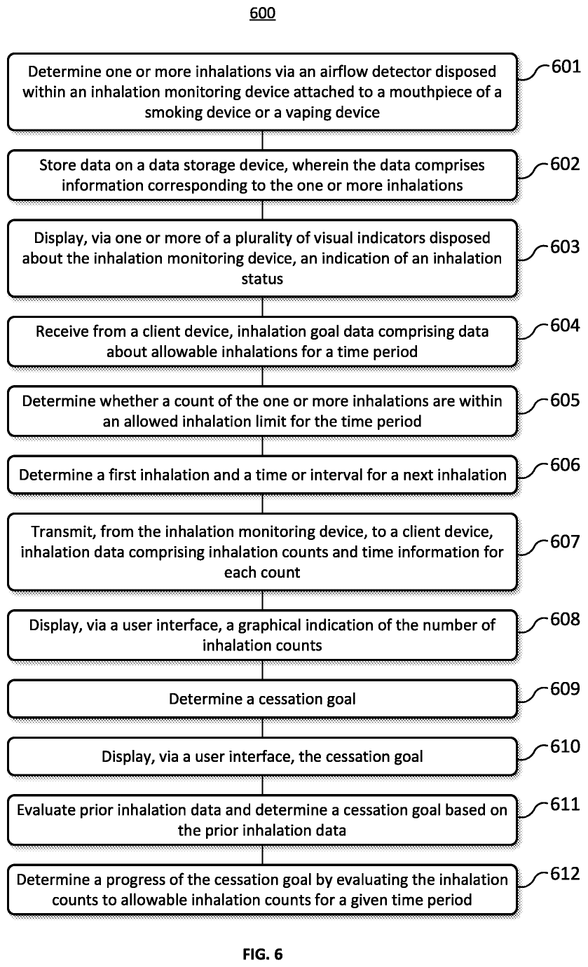

FIG. 5 is a box diagram illustrating an exemplary embodiment of a cessation system 500 and the communication between components of the system in accordance with some embodiments. The exemplary system 500 may comprise cloud AI models 501, a multi-platform application 502, a cloud database 503, a local database 504 and a real-time inhalable substances cessation accessor (cessation device 110).

In some embodiments, the multi-platform application 502 may be initialized with pre-existing user data received from cloud database 503 upon startup. Training and cessation program tuning may be performed between cloud AI models 501 and the multi-platform application 502. Multi-platform application 502 may be configured to transmit cessation program tuning information to the real-time inhalable substances cessation accessory 505. The application may also be configured to send initialization data to the local database 504 for storage. The trigger pressure sensor 506 procedure may be triggered as a result of a user puffing on the cessation accessor 505. This may cause the usage data associated with the puff to be wirelessly sent to the multi-platform application 502. The application may then be configured to transmit real-time puff data to the local database 504 for storage.

Local database 504 may be configured to send to the cloud database 503, the locally stored information received from the multi-platform application 502. The cessation accessory 505 may be configured to receive, from the user, a press of a status button 507. The press of the status button 507 by the user may trigger the setting of one or more indicator lights 508, based on a status of the cessation accessory 505.

FIG. 6 is a flow chart illustrating an exemplary method that may be performed in accordance with some embodiments.

At step 601, the system may determine one or more inhalations via an airflow detector disposed within an inhalation monitoring device attached to a mouthpiece of a smoking device or a vaping device.

At step 602, the system may store data on a data storage device, wherein the data comprises information corresponding to the one or more inhalations.

At step 603, the system may display, via one or more of a plurality of visual indicators disposed about the inhalation monitoring device, an indication of an inhalation status.

At step 604, the system may receive from a client device, inhalation goal data comprising data about allowable inhalations for a time period.

At step 605, the system may determine whether a count of the one or more inhalations are within an allowed inhalation limit for the time period.

At step 606, the system may determine a first inhalation and a time or interval for a next inhalation.

At step 607, the system may transmit, from the inhalation monitoring device, to a client device, inhalation data comprising inhalation counts and time information for each count.

At step 608, the system may display, via a user interface, a graphical indication of the number of inhalation counts.

At step 609, the system may determine a cessation goal.

At step 610, the system may display, via a user interface, the cessation goal.

At step 611, the system may evaluate prior inhalation data and determine a cessation goal based on the prior inhalation data.

At step 612, the system may determine a progress of the cessation goal by evaluating the inhalation counts to allowable inhalation counts for a given time period.

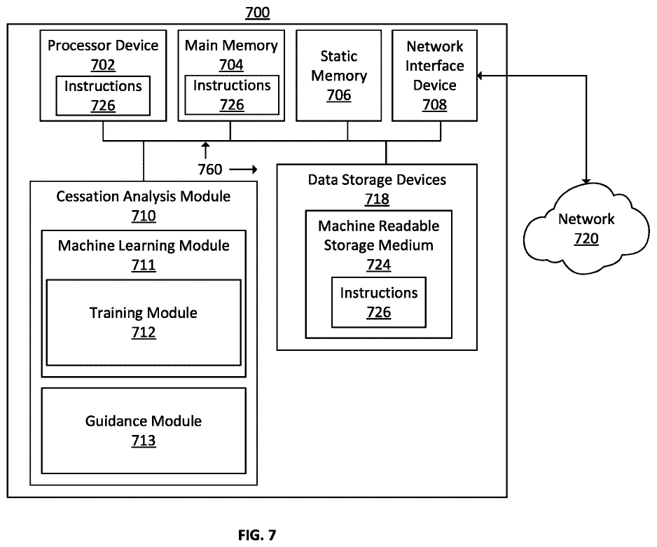

FIG. 7 illustrates an example machine of a computer system within which a set of instructions, for causing the machine to perform any one or more of the methodologies discussed herein, may be executed. In alternative implementations, the machine may be connected (e.g., networked) to other machines in a LAN, an intranet, an extranet, an ad-hoc network, a mesh network, and/or the Internet. The machine may operate in the capacity of a server or a client machine in client-server network environment, as a peer machine in a peer-to-peer (or distributed) network environment, or as a server or a client machine in a cloud computing infrastructure or environment.

The machine may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The example computer system 700 includes a processing device 702, a main memory 704 (e.g., read-only memory (ROM), flash memory, dynamic random access memory (DRAM) such as synchronous DRAM (SDRAM) or Rambus DRAM (RDRAM), etc.), a static memory 706 (e.g., flash memory, static random access memory (SRAM), etc.), and a data storage device 718, which communicate with each other via a bus 760.

Processing device 702 represents one or more general-purpose processing devices such as a microprocessor, a central processing unit, or the like. More particularly, the processing device may be complex instruction set computing (CISC) microprocessor, reduced instruction set computing (RISC) microprocessor, very long instruction word (VLIW) microprocessor, or processor implementing other instruction sets, or processors implementing a combination of instruction sets. Processing device 702 may also be one or more special-purpose processing devices such as an application specific integrated circuit (ASIC), a field programmable gate array (FPGA), a digital signal processor (DSP), network processor, or the like. The processing device 702 is configured to execute instructions 726 for performing the operations and steps discussed herein.

The computer system 700 may further include a network interface device 708 to communicate over the network 720. The computer system 700 also may include cessation analysis module 710. Cessation analysis module 710 may further comprise machine learning module 711 and guidance module 713. Machine learning module 711 may further comprise training module 712. Cessation analysis module 710, machine learning module 711, training module 712 and guidance module 713 may be the same or similar to that of cessation analysis module 205, machine learning module 206, training module 207 and guidance module 208 as disclosed in FIG. 2A.

The data storage device 718 may include a machine-readable storage medium 724 (also known as a computer-readable medium) on which is stored one or more sets of instructions or software 726 embodying any one or more of the methodologies or functions described herein. The instructions 726 may also reside, completely or at least partially, within the main memory 704 and/or within the processing device 702 during execution thereof by the computer system 700, the main memory 704 and the processing device 702 also constituting machine-readable storage media. Information, including data used in the processes and methods of the system and the one or more sets of instructions or software, may also be stored in blockchain, as NFTs or other decentralized technologies.

In one implementation, the instructions 726 include instructions to implement functionality corresponding to the components of a device to perform the disclosure herein. While the machine-readable storage medium 724 is shown in an example implementation to be a single medium, the term “machine-readable storage medium” should be taken to include a single medium or multiple media (e.g., a centralized or distributed database, and/or associated caches and servers) that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media and magnetic media.

It will be appreciated that the present disclosure may include any one and up to all of the following examples.

Example 1. An inhalation monitoring device comprising: a body, the body having a first opening at a first end and a second opening at a second end; an enclosure comprising: an air flow detector; a wireless transceiver; a processor electronically coupled to the wireless transceiver; a data storage device; and a battery; and an attachment mechanism configured to attach the monitoring device to a smoking device or a vaping device; wherein the enclosure is fixed or integral to the body.

Example 2. The inhalation monitoring device of Example 1, wherein the second opening is configured to attach to and create a seal around a mouthpiece of the smoking device or the vaping device.

Example 3. The inhalation monitoring device of Example 2, wherein the attachment mechanism is band connectable to the second end, the attachment band sized to secure the monitoring device to an atomizer cartridge of the smoking device or vaping device.

Example 4. The inhalation monitoring device of any one of Examples 1-3, further comprising: a plurality of visual indicators disposed on the enclosure, the visual indicators comprising light emitting diodes.

Example 5. The inhalation monitoring device of any one of Examples 1-4, wherein the processor is configured to perform the operations of: displaying, via one or more of the plurality of visual indicators, an indication of an inhalation status.

Example 6. The inhalation monitoring device of any one of Examples 1-5, wherein the processor is configured to perform the operations of: determining one or more inhalations via the airflow detector; and storing data on the data storage device about the one or more inhalations.

Example 7. The inhalation monitoring device of any one of Examples 1-6, wherein the processor is configured to perform the operations of: receiving from a client device, inhalation goal data comprising data about allowable inhalations for a time period; and determining whether a count of the one or more inhalations are within an allowed inhalation limit for the time period.

Example 8. The inhalation monitoring device of any one of Examples 1-7, wherein the processor is configured to perform the operations of: determining a first inhalation; and determining a time or interval for a next inhalation.

Example 9. The inhalation monitoring device of any one of Examples 1-8, wherein the processor is configured to perform the operations of: transmitting, via the transceiver, to a client device, inhalation data comprising inhalation counts and time information for each count.

Example 10. The inhalation monitoring device of any one of Examples 1-9, wherein the body has a channel formed therethrough connected to the first opening and the second opening, with the air flow detector positioned about the body to detect air flow through the channel.

Example 11. A method of puff topography monitoring comprising the operations of: determining one or more inhalations via an airflow detector disposed within an inhalation monitoring device attached to a mouthpiece of a smoking device or a vaping device; and storing data on a data storage device, data about the one or more inhalations.

Example 12. The method of Example 11, comprising the operations of: displaying, via one or more of a plurality of visual indicators disposed about the inhalation monitoring device, an indication of an inhalation status.

Example 13. The method of Examples 11-12, comprising the operations of: receiving from a client device, inhalation goal data comprising data about allowable inhalations for a time period; and determining whether a count of the one or more inhalations are within an allowed inhalation limit for the time period.

Example 14. The method of Examples 11-13, comprising the operations of: determining a first inhalation; and determining a time or interval for a next inhalation.

Example 15. The method of Examples 11-14, comprising the operations of: transmitting, from the inhalation monitoring device, to a client device, inhalation data comprising inhalation counts and time information for each count.

Example 16. The method of Examples 11-15, comprising the operations of: displaying, via a user interface, a graphical indication of the number of inhalation counts.

Example 17. The method of Examples 11-16, comprising the operations of: determining a cessation goal; and displaying, via a user interface, the cessation goal.

Example 18. The method of Examples 11-17, comprising the operations of: determining a progress of the cessation goal by evaluating the inhalation counts to allowable inhalation counts for a given time period.

Example 19. The method of Examples 11-18, wherein determining a cessation goal comprises: evaluating prior inhalation data; and determining a cessation goal based on the prior inhalation data.

Example 20. The method of Examples 11-19, comprising the operations of: modifying the cessation goal, wherein the modifying comprises reducing the number of available inhalation counts for the given time period.

Example 21. A inhalable substances cessation system, the system comprising: one or more servers; an application operable on a user device; a monitoring unit, wherein the monitoring unit comprises: an enclosure; a processing unit; a memory module; a datastore module; one or more pressure sensors; a communication module; a battery module; one or more indicators; a mouthpiece; an adapter module; and wherein the monitoring unit is configured to: physically attach to a smoking device or vaping device, wherein the attaching creates an airtight interface between the adapter module of the monitoring unit and the smoking device or vaping device; collect usage data, wherein the collecting comprises: identifying one or more puffs taken by the user, wherein the identifying of puffs is based on detecting changes in pressure at the one or more pressure sensors; recording a timestamp of each of the one or more puffs; recording a duration of each of the one or more puffs; and calculating a volume of each of the one or more puffs based on the duration of the puff and the detected change in pressure at the one or more pressure sensors; generate a user profile, wherein the user profile is based at least in part on usage data collected over a predetermined period of time, and the user profile comprises: an average daily number of puffs; an average puff volume; an average puff duration; an average total daily usage; a maximum flow rate; a flow rate average; and an average time between puffs; calculate, based on the user profile, a goal profile, wherein the goal profile comprises: a goal usage value, wherein the goal usage value corresponds to a total duration of daily use or a total volume of daily use; a number of puffs goal; and a wait time goal, wherein the wait time goal corresponds to a time between puffs.

Example 22. The system of Example 21, wherein the collected usage data is transmitted to the user device, and the user device performs the generation of the user profile and the calculation of the goal profile.

Example 23. The system of any one of Examples 21-22, wherein the user device is configured to display information corresponding to the goal profile and current usage data, wherein the current usage data comprises a current number of puffs, current duration of use and a current usage volume.

Example 24. The system of any one of Examples 21-23, wherein the information displayed on the user device comprises the goal usage value, the current duration of use or the current usage volume, the number of puffs goal, the current number of puffs and a countdown timer, and wherein the countdown timer is configured to display an amount of time until a next puff.

Example 25. The system of any one of Examples 21-24, wherein the countdown timer is reset after detection of each puff.

Example 26. The system of any one of Examples 21-25, wherein the system is further configured to generate a user adherence metric corresponding to how closely the current usage data matches the goal profile.

Example 27. The system of any one of Examples 21-26, wherein the goal profile is updated based on collected usage data over a sliding window.

Example 28. The system of any one of Examples 21-27, wherein the goal profile is calculated by a trained machine learning model.

Example 29. The system of any one of Examples 21-28, wherein the trained machine learning model is a deep learning time series model.

Example 30. The system of any one of Examples 21-29, wherein the trained machine learning model is trained on a training set comprising of collected usage data from one or more users, user profiles of one or more users, goal profiles of one or more users and user adherence metrics of one or more users.

Example 31. The system of any one of Examples 21-30, wherein the one or more indicators of the monitoring unit are light emitting diodes (LEDs).

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. An inhalation monitoring device comprising:
a body, the body having a first opening at a first end and a second opening at a second end;
an enclosure comprising:
an air flow detector;
a wireless transceiver;
a processor electronically coupled to the wireless transceiver;
a data storage device; and
a battery; and
an attachment mechanism configured to attach the monitoring device to a smoking device or a vaping device;
wherein the enclosure is fixed or integral to the body.

2. The device of claim 1, wherein the second opening is configured to attach to and create a seal around a mouthpiece of the smoking device or the vaping device.

3. The device of claim 2, wherein the attachment mechanism is band connectable to the second end, the attachment band sized to secure the monitoring device to an atomizer cartridge of the smoking device or vaping device.

4. The device of claim 2, further comprising:
a plurality of visual indicators disposed on the enclosure, the visual indicators comprising light emitting diodes.

5. The device of claim 3, wherein the processor is configured to perform the operations of:
displaying, via one or more of the plurality of visual indicators, an indication of an inhalation status.

6. The device of claim 1, wherein the processor is configured to perform the operations of:
determining one or more inhalations via the airflow detector; and
storing data on the data storage device about the one or more inhalations.

7. The device of claim 6, wherein the processor is configured to perform the operations of:
receiving from a client device, inhalation goal data comprising data about allowable inhalations for a time period; and
determining whether a count of the one or more inhalations are within an allowed inhalation limit for the time period.

8. The device of claim 1, wherein the processor is configured to perform the operations of:
determining a first inhalation; and
determining a time or interval for a next inhalation.

9. The device of claim 1, wherein the processor is configured to perform the operations of:
transmitting, via the transceiver, to a client device, inhalation data comprising inhalation counts and time information for each count.

10. The device of claim 1, wherein the body has a channel formed therethrough connected to the first opening and the second opening, with the air flow detector positioned about the body to detect air flow through the channel.

11. A method of puff topography monitoring comprising the operations of:
determining one or more inhalations via an airflow detector disposed within an inhalation monitoring device attached to a mouthpiece of a smoking device or a vaping device; and
storing data on a data storage device, data about the one or more inhalations.

12. The method of claim 11, comprising the operations of:
displaying, via one or more of a plurality of visual indicators disposed about the inhalation monitoring device, an indication of an inhalation status.

13. The method of claim 11, comprising the operations of:
receiving from a client device, inhalation goal data comprising data about allowable inhalations for a time period; and
determining whether a count of the one or more inhalations are within an allowed inhalation limit for the time period.

14. The method of claim 11, comprising the operations of:
determining a first inhalation; and
determining a time or interval for a next inhalation.

15. The method of claim 11, comprising the operations of:
transmitting, from the inhalation monitoring device, to a client device, inhalation data comprising inhalation counts and time information for each count.

16. The method of claim 16, comprising the operations of:
displaying, via a user interface, a graphical indication of the number of inhalation counts.

17. The method of claim 16, comprising the operations of:
determining a cessation goal; and
displaying, via a user interface, the cessation goal.

18. The method of claim 17, comprising the operations of:
determining a progress of the cessation goal by evaluating the inhalation counts to allowable inhalation counts for a given time period.

19. The method of claim 18, wherein determining a cessation goal comprises:
evaluating prior inhalation data; and
determining a cessation goal based on the prior inhalation data.

20. The method of claim 18, comprising the operations of:
modifying the cessation goal, wherein the modifying comprises reducing the number of available inhalation counts for the given time period.

21. A inhalable substances cessation system, the system comprising:
one or more servers;
an application operable on a user device;
a monitoring unit, wherein the monitoring unit comprises:
an enclosure;
a processing unit;
a memory module;
a datastore module;
one or more pressure sensors;
a communication module;
a battery module;
one or more indicators;
a mouthpiece;
an adapter module; and
wherein the monitoring unit is configured to:
physically attach to a smoking device or vaping device, wherein the attaching creates an airtight interface between the adapter module of the monitoring unit and the smoking device or vaping device;
collect usage data, wherein the collecting comprises:
identifying one or more puffs taken by the user, wherein the identifying of puffs is based on detecting changes in pressure at the one or more pressure sensors;
recording a timestamp of each of the one or more puffs;
recording a duration of each of the one or more puffs; and
calculating a volume of each of the one or more puffs based on the duration of the puff and the detected change in pressure at the one or more pressure sensors;
generate a user profile, wherein the user profile is based at least in part on usage data collected over a predetermined period of time, and the user profile comprises:
an average daily number of puffs;
an average puff volume;
an average puff duration;
an average total daily usage;
a maximum flow rate;
a flow rate average; and
an average time between puffs;
calculate, based on the user profile, a goal profile, wherein the goal profile comprises:
a goal usage value, wherein the goal usage value corresponds to a total duration of daily use or a total volume of daily use;
a number of puffs goal; and
a wait time goal, wherein the wait time goal corresponds to a time between puffs.

22. The system of claim 21, wherein the collected usage data is transmitted to the user device, and the user device performs the generation of the user profile and the calculation of the goal profile.

23. The system of claim 22, wherein the user device is configured to display information corresponding to the goal profile and current usage data, wherein the current usage data comprises a current number of puffs, current duration of use and a current usage volume.

24. The system of claim 23, wherein the information displayed on the user device comprises the goal usage value, the current duration of use or the current usage volume, the number of puffs goal, the current number of puffs and a countdown timer, and wherein the countdown timer is configured to display an amount of time until a next puff.

25. The system of claim 24, wherein the countdown timer is reset after detection of each puff.

26. The system of claim 24, wherein the system is further configured to generate a user adherence metric corresponding to how closely the current usage data matches the goal profile.

27. The system of claim 26, wherein the goal profile is updated based on collected usage data over a sliding window.

28. The system of claim 27, wherein the goal profile is calculated by a trained machine learning model.

29. The system of claim 28, wherein the trained machine learning model is a deep learning time series model.

30. The system of claim 28, wherein the trained machine learning model is trained on a training set comprising of collected usage data from one or more users, user profiles of one or more users, goal profiles of one or more users and user adherence metrics of one or more users.

31. The system of claim 30, wherein the one or more indicators of the monitoring unit are light emitting diodes (LEDs).
